(19) United States
(12) Patent Application Publication
(10) Pub. No.: US 2020/0339562 A1
(43) Pub. Date: Oct. 29, 2020

(54) COVALENT BTK INHIBITORS AND USES THEREOF

(71) Applicant: X-Chem, Inc., Waltham, MA (US)

(72) Inventors: Matthew CLARK, Lexington, MA (US); Anthony D. KEEFE, Cambridge, MA (US); John GUILINGER, Wakefield, MA (US); Ying ZHANG, Lexington, MA (US); Xia TIAN, Cambridge, MA (US)

(21) Appl. No.: 16/097,093

(22) PCT Filed: Apr. 28, 2017
(86) PCT No.: PCT/US2017/030185

§ 371 Date: Oct. 26, 2018

Related U.S. Application Data
(60) Provisional application No. 62/329,708, filed on Apr. 29, 2016.

(51) Int. Cl. C07D 471/04; C07D 209/42; C07D 207/16; C07D 277/56; C07D 303/48

(52) U.S. Cl. CPC C07D 471/04; C07D 209/42; C07D 303/48; C07D 277/56; C07D 207/16

(57) ABSTRACT

The present invention features compounds having BTK inhibitory activity. The compounds of the invention, alone or in combination with other pharmaceutically active agents, can be used for treating or preventing various medical conditions, such as cardiovascular diseases, respiratory diseases, inflammation, and diabetes.

### BACKGROUND OF THE INVENTION

The present invention relates to compounds having kinase inhibitory activity, as well as their therapeutic, diagnostic, and medical uses.

Bruton agammaglobulinemia tyrosine kinase (Btk or BTK) is a cytoplasmic kinase in the Tec family. Btk plays an important role in the development and regulation of lymphoid, myeloid, and mast cell lineages, such as by activating the B-cell receptor (BCR) signaling pathway, mediating cytokine receptor signaling, and participating in mast cell activation. However, activation or overactivation of Btk can contribute to or promote numerous diseases, including B-cell malignancies (e.g., Hodgkin's lymphoma, non-Hodgkin lymphoma, or chronic lymphocytic leukemia), inflammatory or autoimmune disorders (e.g., rheumatoid arthritis, systemic lupus erythematosus, or multiple sclerosis), and mast cell malignancies (e.g., pancreatic insulinoma). Thus, there is a need for new compounds that inhibit Btk and treatment methods using such compounds.

### SUMMARY OF THE INVENTION

In one aspect, the invention features a compound having the structure of Formula I:

A—B—C Formula I

wherein A is

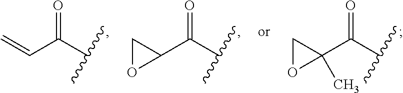

B has the structure:

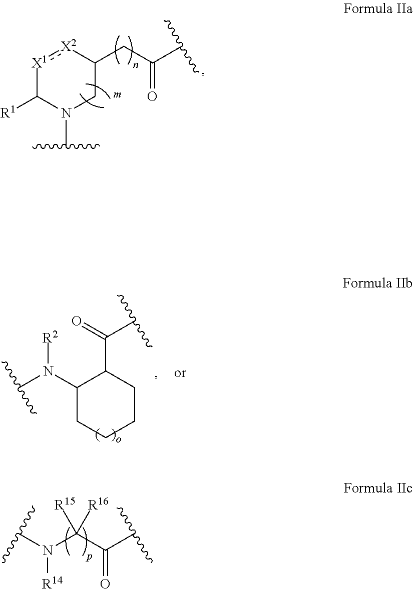

wherein B is conjugated to A at the nitrogen and to C at the carbonyl;

the dotted line is an optional double bond;

m is 0 or 1;

n is 0, 1, or 2;

o is 0 or 1;

p is 1 or 2;

R¹ is hydrogen or combines with R³ or R⁴ to form a 5- to 6-membered carbocyclic ring;

R² is hydrogen or C₁-C₆ alkyl;

X¹ is S or CR³R⁴, wherein R³ and R⁴ are each, independently, hydrogen or combine with R¹ to form a 5- to 6-membered carbocyclic ring, wherein if a double bond is present, then R⁴ is absent; and

X² is CR⁵R⁶, wherein R⁵ and R⁶ are each, independently, hydrogen or C₁-C₆ alkyl, wherein if a double bond is present, R⁶ is absent;

R¹⁴ is hydrogen or C₁-C₆ alkyl;

each R¹⁵ and R¹⁶ is, independently, optionally substituted C₁-C₆ heteroalkyl, optionally substituted C₆-C₁₀ aryl; or optionally substituted C₁-C₆ alkyl C₆-C₁₀ aryl; and

C has the structure:

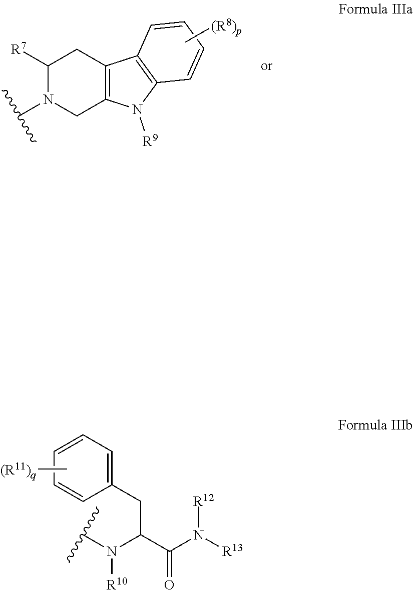

wherein p is 0, 1, 2, 3, or 4;

q is 0, 1, 2, 3, 4, or 5;

R⁷ is hydrogen or amido;

each R⁸ and R¹¹ is, independently hydrogen, hydroxy, C₁-C₆ alkyl, cyano, or halo; and

R⁹, R¹⁰, R¹², and R¹³ are each, independently, hydrogen or C₁-C₆ alkyl.

In some embodiments, B has the structure of Formula IIa:

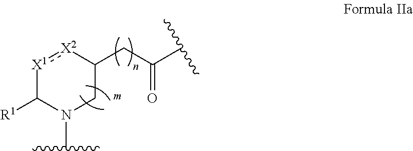

wherein B is conjugated to A at the nitrogen and to C at the carbonyl;

the dotted line is an optional double bond;

m is 0 or 1;

n is 0, 1, or 2;

R¹ is hydrogen or combines with R³ or R⁴ to form a 5- to 6-membered carbocyclic ring;

X¹ is S or CR³R⁴, wherein R³ and R⁴ are each, independently, hydrogen or combine with R¹ to form a 5- to 6-membered carbocyclic ring, wherein if a double bond is present then R⁴ is absent; and

X² is CR⁵R⁶, wherein R⁵ and R⁶ are each, independently, hydrogen or C₁-C₆ alkyl, wherein if a double bond is present, R⁶ is absent.

In some embodiments, m is 1. In some embodiments, R¹ is hydrogen. In some embodiments, X¹ is CR³R⁴ such as wherein R³ and R⁴ are both hydrogen. In some embodiments, X² is CR⁵R⁶ such as wherein R⁵ and R⁶ are both hydrogen. In some embodiments, n is 1.

In some embodiments, B has the structure:

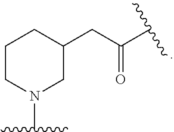 [SMILES: *C(=O)CC1CCCN(*)C1]

In some embodiments, B has the structure:

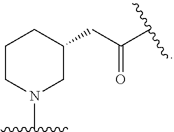 [SMILES: *C(=O)C[C@H]1CCCN(*)C1]

In some embodiments, m is 0. In some embodiments, n is 2. In some embodiments, R¹ is hydrogen. In some embodiments, X¹ is CR³R⁴ such as wherein R³ and R⁴ are both hydrogen. In some embodiments, X² is CR⁵R⁶ such as wherein R⁵ and R⁶ are both hydrogen.

In some embodiments, B has the structure:

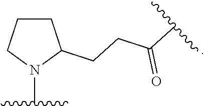 [SMILES: *C(=O)CCC1CCCN1*]

In some embodiments, n is 0. In some embodiments, R¹ is hydrogen. In some embodiments, X¹ is S. In some embodiments, X² is CR⁵R⁶ such as wherein R⁵ and R⁶ are both methyl.

In some embodiments, B has the structure:

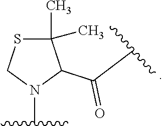 [SMILES: *C(=O)C1N(*)CSC1(C)C]

In some embodiments, B has the structure:

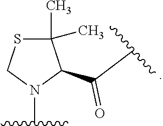 [SMILES: *C(=O)[C@H]1N(*)CSC1(C)C]

In some embodiments, B has the structure:

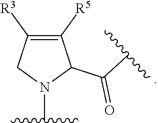

In some embodiments, R³ and R⁵ are both hydrogen.

In some embodiments, X² is CR⁵R⁶ such as wherein R⁵ and R⁶ are both hydrogen. In some embodiments, X¹ is CR³R⁴. In some embodiments, R⁴ is hydrogen. In some embodiments, R¹ and R³ combine to form a 5- or 6-membered carbocyclic ring.

In some embodiments, B has the structure:

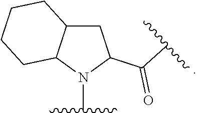 [SMILES: *C(=O)C1CC2CCCCC2N1*]

In some embodiments, B has the structure:

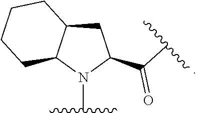 [SMILES: *C(=O)[C@@H]1C[C@@H]2CCCC[C@@H]2N1*]

In some embodiments, B has the structure of Formula IIb:

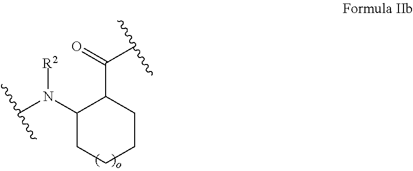

wherein B is conjugated to A at the nitrogen and to C at the carbonyl;

o is 0 or 1; and

R² is hydrogen or C₁-C₆ alkyl.

In some embodiments, o is 0. In some embodiments, R² is hydrogen.

In some embodiments, B has the structure of Formula IIc:

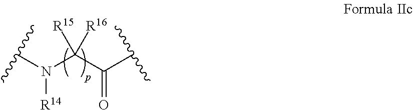

wherein B is conjugated to A at the nitrogen and to C at the carbonyl;

p is 1 or 2;

R¹⁴ is hydrogen or C₁-C₆ alkyl; and

each R¹⁵ and R¹⁶ is, independently, optionally substituted C₁-C₆ heteroalkyl, optionally substituted C₆-C₁₀ aryl; or optionally substituted C₁-C₆ alkyl C₆-C₁₀ aryl.

In some embodiments, R¹⁴ is hydrogen.

In some embodiments, p is 1. In some embodiments, R¹⁵ is hydrogen. In some embodiments, R¹⁶ is optionally substituted C₆-C₁₀ aryl (e.g., phenyl). In some embodiments, R¹⁶ is optionally substituted C₁-C₆ heteroalkyl

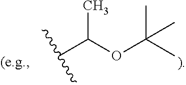 [SMILES: *C(C)OC(C)(C)C]

In some embodiments, B has the structure:

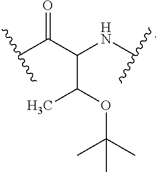 [SMILES: *NC(C(*)=O)C(C)OC(C)(C)C]

In some embodiments, B has the structure:

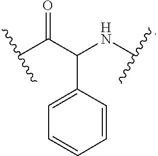 [SMILES: *NC(C(*)=O)c1ccccc1]

In some embodiments, p is 2. In some embodiments, R¹⁵ is hydrogen. In some embodiments, R¹⁶ is optionally substituted C₁-C₆ alkyl C₆-C₁₀ aryl (e.g., 2-fluoro-benzyl).

In some embodiments, B has the structure:

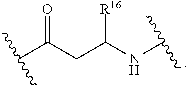

In some embodiments, B has the structure:

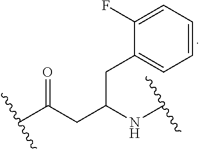 [SMILES: *NC(CC(*)=O)Cc1ccccc1F]

In some embodiments of any of the foregoing compounds, C has the structure of Formula IIIa:

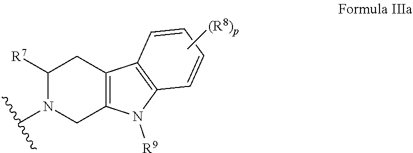

wherein p is 0, 1, 2, 3, or 4;

R⁷ is hydrogen or amido;

each R⁸ is, independently hydrogen, hydroxy, C₁-C₆ alkyl, cyano, or halo; and

R⁹ is hydrogen or C₁-C₆ alkyl.

In some embodiments, p is 0. In some embodiments, R⁹ is hydrogen. In some embodiments, R⁷ is amido.

In some embodiments, C has the structure:

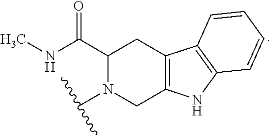 [SMILES: *N1Cc2[nH]c3ccccc3c2CC1C(=O)NC]

In some embodiments, C has the structure:

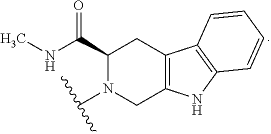 [SMILES: *N1Cc2[nH]c3ccccc3c2C[C@@H]1C(=O)NC]

In some embodiments, C has the structure:

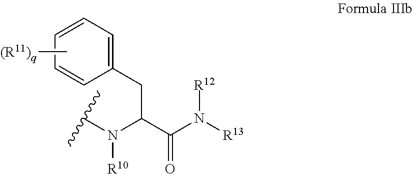

wherein q is 0, 1, 2, 3, 4, or 5;

each R¹¹ is, independently hydrogen, hydroxy, C₁-C₆ alkyl, cyano, or halo; and

R¹² and R¹³ are each, independently, hydrogen or C₁-C₆ alkyl.

In some embodiments, R¹⁰ is hydrogen. In some embodiments, R¹² is hydrogen. In some embodiments, R¹³ is C₁-C₆ alkyl (e.g., methyl). In some embodiments, q is 1.

In some embodiments, C has the structure:

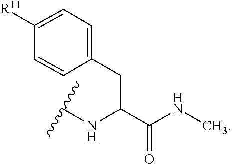

In some embodiments, C has the structure:

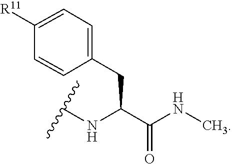

In some embodiments, R¹¹ is cyano.

In some embodiments of any of the foregoing compounds, A is

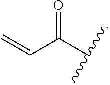 [SMILES: *C(=O)C=C]

In some embodiments of any of the foregoing compounds, A is

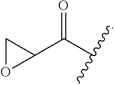 [SMILES: *C(=O)C1CO1]

In some embodiments of any of the foregoing compounds, A is

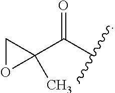 [SMILES: *C(=O)C1(C)CO1]

In another aspect, the invention features a compound having the structure of any one of compounds 1-11 in Table 1.

In some embodiments, the compound of the invention has an IC₅₀ value less than about 1.0 μM (e.g., less than about 0.9 μM, less than about 0.8 μM, less than about 0.5 μM, less than about 0.3 μM, less than about 0.2 μM, less than about 0.1 μM, less than about 0.09 μM, less than about 0.08 μM, less than about 0.05 μM, less than about 0.04 μM, less than about 0.03 μM, less than about 0.025 μM, less than about 0.015 μM, less than about 0.01 μM, less than about 0.005 μM, less than about 0.002 μM, less than about 0.0015 μM, or less than about 0.001 μM). In some embodiments, the compound has an IC₅₀ value from about 0.0001 μM to about 0.9 μM (e.g., from about 0.0001 μM to about 0.8 μM, from about about 0.0001 μM to about 0.5 μM, from about 0.0001 μM to about 0.3 μM, from about 0.0001 μM to about 0.2 μM, from about 0.0001 μM to about 0.1 μM, from about 0.0001 μM to about 0.09 μM, from about 0.0001 μM to about 0.08 μM, from about 0.0001 μM to about 0.05 μM, from about 0.0001 μM to about 0.04 μM, from about 0.0001 μM to about 0.03 μM, from about 0.0001 μM to about 0.025 μM, from about 0.0001 μM to about 0.015 μM, from about 0.0001 μM to about 0.01 μM, from about 0.0001 μM to about 0.005 μM, 0.0002 μM to about 0.9 μM, from about 0.0002 μM to about 0.8 μM, from about 0.0002 μM to about 0.5 μM, from about 0.0002 μM to about 0.3 μM, from about 0.0002 μM to about 0.2 μM, from about 0.0002 μM to about 0.1 μM, from about 0.0002 μM to about 0.09 μM, from about 0.0002 μM to about 0.08 μM, from about 0.0002 μM to about 0.05 μM, from about 0.0002 μM to about 0.04 μM, from about 0.0002 μM to about 0.03 μM, from about 0.0002 μM to about 0.025 μM, from about 0.0002 μM to about 0.015 μM, from about 0.0002 μM to about 0.01 μM, from about 0.0002 μM to about 0.005 μM, about 0.0005 μM to about 0.9 μM, from about 0.0005 μM to about 0.8 μM, from about 0.0005 μM to about 0.5 μM, from about 0.0005 μM to about 0.3 μM, from about 0.0005 μM to about 0.2 μM, from about 0.0005 μM to about 0.1 μM, from about 0.0005 μM to about 0.09 μM, from about 0.0005 μM to about 0.08 μM, from about 0.0005 μM to about 0.05 μM, from about 0.0005 μM to about 0.04 μM, from about 0.0005 μM to about 0.03 μM, from about 0.0005 μM to about 0.025 μM, from about 0.0005 μM to about 0.015 μM, from about 0.0005 μM to about 0.01 μM, from about 0.0005 μM to about 0.005 μM, from about 0.0005 μM to about 0.002 μM, from about 0.0005 μM to about 0.0015 μM, or from about 0.0005 μM to about 0.001 μM). In some embodiments, the compound has an IC₅₀ value from about 0.02 μM to about 1.0 μM (e.g., from about 0.02 μM to about 0.9 μM, from about 0.02 μM to about 0.75 μM, from about 0.02 μM to about 0.5 μM, from about 0.02 μM to about 0.3 μM, from about 0.02 μM to about 0.25 μM, from about 0.02 μM to about 0.2 μM, from about 0.02 μM to about 0.15 μM, from about 0.02 μM to about 0.1 μM, from about 0.02 μM to about 0.09 μM, from about 0.02 μM to about 0.08 μM, from about 0.02 μM to about 0.05 μM, from about 0.02 μM to about 0.04 μM, from about 0.02 μM to about 0.03 μM, or from about 0.02 μM to about 0.025 μM).

In another aspect, the invention features a pharmaceutical composition including any of the foregoing compounds and a pharmaceutically acceptable excipient.

In another aspect, the invention features a method of inhibiting Bruton's tyrosine kinase, the method including contacting a cell with any of the foregoing compounds.

In another aspect, the invention features a method of treating a B-cell associated disease or a mast cell associated disease (e.g., cancer, an inflammatory disorder, or an autoimmune disorder) in a subject in need thereof, the method including administering an effective amount of any of the foregoing compounds or pharmaceutically acceptable salts thereof, or any of the foregoing compositions to the subject.

In another aspect, the invention features a method of treating cancer (e.g., leukemia, lymphoma, myeloma, or a pancreatic neoplasm such as non-Hodgkin lymphoma, B-cell lymphoma, chronic lymphocytic leukemia, small lymphocytic lymphoma, pancreatic insulinoma, pancreatic glucagonoma, or pancreatic gastrinoma) in a subject in need thereof, said method including administering an effective amount of any of the foregoing compounds or pharmaceutically acceptable salts thereof, or any of the foregoing compositions to the subject.

In another aspect, the invention features a method of treating an inflammatory or autoimmune disorder (e.g., rheumatoid arthritis, systemic lupus erythematosus, multiple sclerosis, idiopathic thrombocytopenic purpura, glomerulonephritis, autoimmune-mediated hemolytic anemia, immune complex mediated vasculitis, or psoriasis) in a subject in need thereof, the method including administering an effective amount of any of the foregoing compounds or pharmaceutically acceptable salts thereof, or any of the foregoing compositions to the subject.

Non-limiting exemplary cancers include leukemia, including acute myeloid leukemia (AML), acute lymphocytic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), and B-cell prolymphocytic leukemia (B-PLL); lymphomas, including Hodgkin and non-Hodgkin lymphoma, such as B-cell lymphomas (e.g., diffuse large B-cell lymphoma (e.g., mediastinal (thymic) large B-cell lymphoma and intravascular large B-cell lymphoma), follicular lymphoma, small lymphocytic lymphoma (SLL), chronic lymphocytic leukemia/small lymphocytic lymphoma (CLL/SLL), mantle cell lymphoma (e.g., relapsed or refractory), marginal zone B-cell lymphomas, Burkitt lymphoma, lymphoplasmacytic lymphoma, hairy cell leukemia, primary central nervous system (CNS) lymphoma, primary effusion lymphoma, and lymphomatoid granulomatosis); myelomas, including multiple myeloma, plasmacytoma, localized myeloma, and extramedullary myeloma; and other cancers, such as pancreatic neoplasms, including pancreatic exocrine tumors (e.g., ductal adenocarcinoma, signet ring cell carcinomas, hepatoid carcinomas, colloid carcinomas, undifferentiated carcinomas, and undifferentiated carcinomas with osteoclast-like giant cells), pancreatic cystic neoplasms (e.g., mucinous cystadenoma, serous cystadenoma, and mucinous ductal ectasia), pancreatic neuroendocrine tumors (e.g., insulinoma, glucagonoma, gastrinoma, VIPoma, and somatostatinoma), papillary cystic neoplasms of the pancreas, lymphoma of the pancreas, and acinar cell tumors of the pancreas, or any described herein.

Non-limiting exemplary inflammatory or autoimmune disorders include autoimmune arthritis (e.g., rheumatoid arthritis, psoriatic arthritis, ankylosing spondylitis, Still's disease, juvenile arthritis, and mixed and undifferentiated connective tissue diseases), autoimmune hemolytic and thrombocytopenic states (e.g., autoimmune-mediated hemolytic anemia, e.g., warm autoimmune hemolytic anemia, cold autoimmune hemolytic anemia, cold agglutinin disease, and paroxysmal cold hemoglobinuria), autoimmune hepatitis, Behçet's disease, chronic idiopathic thrombocytopenic purpura (ITP), glomerulonephritis, Goodpasture's syndrome (and associated glomerulonephritis and pulmonary hemorrhage), idiopathic thrombocytopenic purpura (ITP) (e.g., acute ITP or chronic ITP), inflammatory bowel disease (including Crohn's disease and ulcerative colitis), multiple sclerosis, psoriasis (including psoriatic lesions in the skin), systemic lupus erythematosus (and associated glomerulonephritis), and vasculitis (including antineutrophil cytoplasmic antibodies-associated vasculitis, immune complex mediated vasculitis, and Wegener's granulomatosis), or any described herein.

### Chemical Terms

It is to be understood that the terminology employed herein is for the purpose of describing particular embodiments and is not intended to be limiting.

The term “acyl,” as used herein, represents a hydrogen or an alkyl group, as defined herein, that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amido,” as used herein, represents —C(O)N(R^{N1})₂, wherein each R^{N1} is, independently, H, OH, NO₂, N(R^{N2})₂, SO₂ORN², SO₂RN², SORN², an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “amino,” as used herein, represents —N(R^{N1})₂, wherein each R^{N1} is, independently, H, OH, NO₂, N(R^{N2})₂, SO₂ORN², SO₂RN², SORN², an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1 H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-6 alkyl C₆₋₁₀ aryl, C₁₋₁₀ alkyl C₆₋₁₀ aryl, or C₁₋₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “carbonyl,” as used herein, refers to a —O(O)— group.

The term “cyano,” as used herein, represents a —CN group.

The terms “carbocyclyl,” as used herein, refer to a non-aromatic C₃₋₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O- (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O-. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O-. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁₋₆ alkyl C₂₋₉ heteroaryl, C₁₋₁₀ alkyl C₂₋₉ heteroaryl, or C₁₋₂₀ alkyl C₂₋₉ heteroaryl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁₋₆ alkyl C₂₋₉ heterocyclyl, C₁₋₁₀ alkyl C₂₋₉ heterocyclyl, or C₁₋₂₀ alkyl C₂₋₉ heterocyclyl). In some embodiments, the akyl and the heterocyclyl each can be further substituted with 1, 2, 3, or 4 substituent groups as defined herein for the respective groups.

The term “hydroxy,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH2 or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

Other features and advantages of the invention will be apparent from the following detailed description, the drawings, and the claims.

### DETAILED DESCRIPTION OF THE INVENTION

### Compounds

The invention features novel compounds of Formula I:

A—B—C Formula I

wherein A is

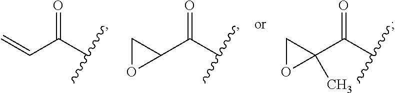

B has the structure:

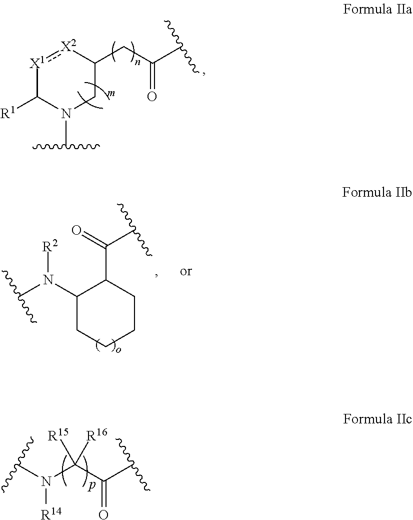

wherein B is conjugated to A at the nitrogen and to C at the carbonyl;

the dotted line is an optional double bond;

m is 0 or 1;

n is 0, 1, or 2;

o is 0 or 1;

p is 1 or 2;

R¹ is hydrogen or combines with R³ or R⁴ to form a 5- to 6-membered carbocyclic ring;

R² is hydrogen or C₁-C₆ alkyl;

X¹ is S or CR³R⁴, wherein R³ and R⁴ are each, independently, hydrogen or combine with R¹ to form a 5- to 6-membered carbocyclic ring, wherein if a double bond is present, then R⁴ is absent; and

X² is CR⁵R⁶, wherein R⁵ and R⁶ are each, independently, hydrogen or C₁-C₆ alkyl, wherein if a double bond is present, R⁶ is absent;

R¹⁴ is hydrogen or C₁-C₆ alkyl;

each R¹⁵ and R¹⁶ is, independently, optionally substituted C₁-C₆ heteroalkyl, optionally substituted C₆-C₁₀ aryl; or optionally substituted C₁-C₆ alkyl C₆-C₁₀ aryl; and

C has the structure:

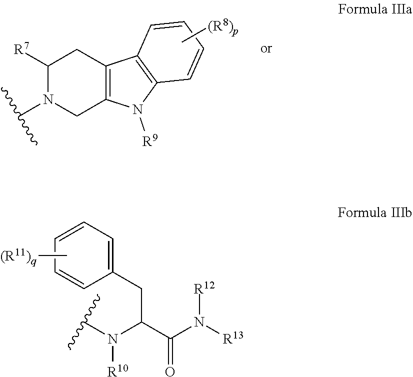

wherein p is 0, 1, 2, 3, or 4;

q is 0, 1, 2, 3, 4, or 5;

R⁷ is hydrogen or amido;

each R⁸ and R¹¹ is, independently hydrogen, hydroxy, C₁-C₆ alkyl, cyano, or halo; and

R⁹, R¹⁰, R¹², and R¹³ are each, independently, hydrogen or C₁-C₆ alkyl.

In some embodiments, the compounds have kinase (e.g., BTK) inhibitory activity. In some embodiments, the compounds may be useful in pharmaceutical and diagnostic compositions containing them and medical uses. Exemplary compounds of the invention are shown in Table 1, including stereoisomers (e.g., diastereomers or enantiomers), or pharmaceutically acceptable salts thereof.

TABLE 1
Exemplary Compounds of the Invention

| Compound | |
|---|---|
| # | Structure |
| 1 | 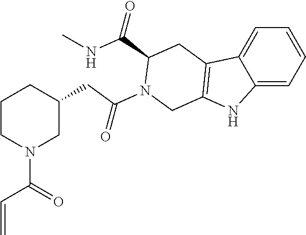 [SMILES: C=CC(=O)N1CCC[C@H](CC(=O)N2Cc3[nH]c4ccccc4c3C[C@@H]2C(=O)NC)C1] |
| 2 | 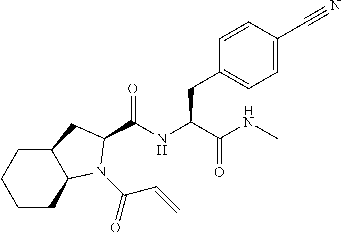 [SMILES: C=CC(=O)N1[C@H](C(=O)N[C@@H](Cc2ccc(C#N)cc2)C(=O)NC)C[C@@H]2CCCC[C@@H]21] |
| 3 | 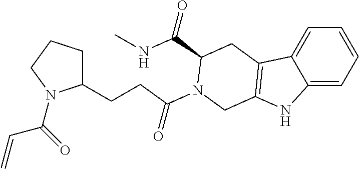 [SMILES: C=CC(=O)N1CCCC1CCC(=O)N1Cc2[nH]c3ccccc3c2C[C@@H]1C(=O)NC] |
| 4 | 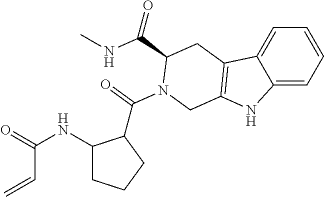 [SMILES: C=CC(=O)NC1CCCC1C(=O)N1Cc2[nH]c3ccccc3c2C[C@@H]1C(=O)NC] |
| 5 | 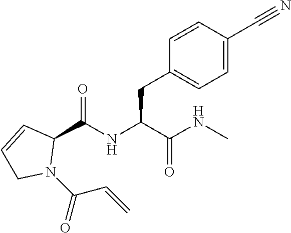 [SMILES: C=CC(=O)N1CC=C[C@H]1C(=O)N[C@@H](Cc1ccc(C#N)cc1)C(=O)NC] |
| 6 | 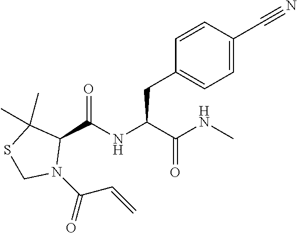 [SMILES: C=CC(=O)N1CSC(C)(C)[C@H]1C(=O)N[C@@H](Cc1ccc(C#N)cc1)C(=O)NC] |
| 7 | 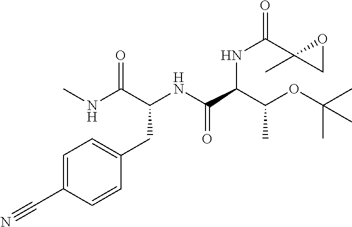 [SMILES: CNC(=O)[C@@H](Cc1ccc(C#N)cc1)NC(=O)[C@@H](NC(=O)[C@]1(C)CO1)[C@@H](C)OC(C)(C)C] |
| 8 | 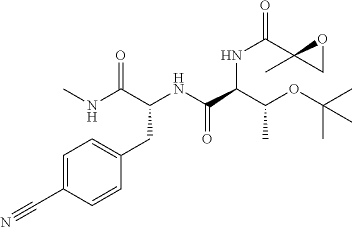 [SMILES: CNC(=O)[C@@H](Cc1ccc(C#N)cc1)NC(=O)[C@@H](NC(=O)[C@@]1(C)CO1)[C@@H](C)OC(C)(C)C] |
| 9 | 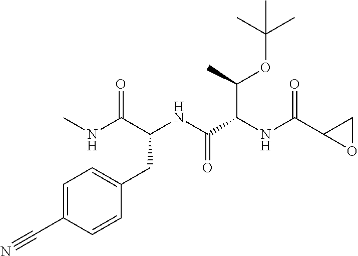 [SMILES: CNC(=O)[C@@H](Cc1ccc(C#N)cc1)NC(=O)[C@@H](NC(=O)C1CO1)[C@@H](C)OC(C)(C)C] |
| 10 | 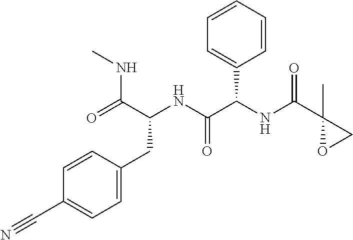 [SMILES: CNC(=O)[C@@H](Cc1ccc(C#N)cc1)NC(=O)[C@@H](NC(=O)[C@@]1(C)CO1)c1ccccc1] |
| 11 | 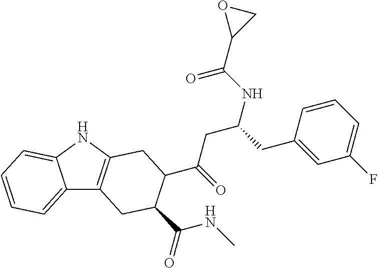 [SMILES: CNC(=O)[C@H]1Cc2c([nH]c3ccccc23)CC1C(=O)C[C@@H](Cc1cccc(F)c1)NC(=O)C1CO1] |

Exemplary methods for synthesizing compounds of the invention are described herein.

### Pharmaceutical Uses

The compounds described herein are useful in the methods of the invention and, while not bound by theory, are believed to exert their desirable effects through their ability to inhibit the activity of BTK. The compounds of the invention having useful BTK inhibiting activity, may be useful to treat, prevent, or reduce the risk of, diseases or conditions that are ameliorated by a reduction in BTK activity, such as a B-cell related disorder or a mast cell related disorder (e.g., any disorder described herein).

Cancer

BTK is a key regulator in B-cell development, differentiation, and signaling, as well as in mast cell activation. Accordingly, activation of BTK has been implicated in the pathology of numerous proliferative disorders, including B-cell, mast cell, and other non-B-cell associated cancers.

Exemplary proliferative disorders (e.g., cancers) include leukemia, including acute myeloid leukemia (AML), acute lymphocytic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), and B-cell prolymphocytic leukemia (B-PLL); lymphomas, including Hodgkin and non-Hodgkin lymphoma, such as B-cell lymphomas (e.g., diffuse large B-cell lymphoma (e.g., mediastinal (thymic) large B-cell lymphoma and intravascular large B-cell lymphoma), follicular lymphoma, small lymphocytic lymphoma (SLL), chronic lymphocytic leukemia/small lymphocytic lymphoma (CLL/SLL), mantle cell lymphoma (e.g., relapsed or refractory), marginal zone B-cell lymphomas (e.g., extranodal marginal zone B-cell lymphoma, nodal marginal zone B-cell lymphoma, and splenic marginal zone lymphoma), Burkitt lymphoma, lymphoplasmacytic lymphoma (Waldenstrom macroglobulinemia), hairy cell leukemia, primary central nervous system (CNS) lymphoma, primary effusion lymphoma, and lymphomatoid granulomatosis); myelomas, including multiple myeloma (plasma cell myeloma), plasmacytoma, localized myeloma, and extramedullary myeloma; and other cancers, such as pancreatic neoplasms, including pancreatic exocrine tumors (e.g., ductal adenocarcinoma, signet ring cell carcinomas, hepatoid carcinomas, colloid carcinomas, undifferentiated carcinomas, and undifferentiated carcinomas with osteoclast-like giant cells), pancreatic cystic neoplasms (e.g., mucinous cystadenoma, serous cystadenoma, and mucinous ductal ectasia), pancreatic neuroendocrine tumors (e.g., insulinoma, glucagonoma, gastrinoma (Zollinger-Ellison syndrome), VIPoma, and somatostatinoma), papillary cystic neoplasms of the pancreas, lymphoma of the pancreas, and acinar cell tumors of the pancreas; malignant glioma; and papillary thyroid cancer.

Inflammatory Disorders (Including Autoimmune Disorders)

Inhibition of BTK has been shown to mitigate inflammation and/or suppress the production of inflammatory cytokines. Accordingly, the compounds of the invention can be used to treat or prophylactically treat inflammatory disorders, including autoimmune disorders.

Exemplary inflammatory or autoimmune disorders include rheumatoid arthritis, systemic lupus erythematosus (and associated glomerulonephritis), multiple sclerosis, and asthma. Further exemplary disorders include acute disseminated encephalomyelitis, Addison's disease, allergy, alopecia universalis, Alzheimer's disease, ankylosing spondylitis, antiphospholipid antibody syndrome, aplastic anemia, appendicitis, atherosclerosis, autoimmune arthritis (e.g., rheumatoid arthritis, psoriatic arthritis, ankylosing spondylitis, Still's disease, juvenile arthritis, and mixed and undifferentiated connective tissue diseases), autoimmune hemolytic and thrombocytopenic states (e.g., autoimmune-mediated hemolytic anemia, e.g., warm autoimmune hemolytic anemia, cold autoimmune hemolytic anemia, cold agglutinin disease, and paroxysmal cold hemoglobinuria), autoimmune hepatitis, Behçet's disease, blepharitis, bronchiolitis, bronchitis, bursitis, celiac disease, cervicitis, cholangitis, cholecystitis, chronic fatigue, chronic idiopathic thrombocytopenic purpura (ITP), colitis, conjunctivitis, Crohn's disease, cystitis, dacryoadenitis, dermatitis (including contact dermatitis), dermatomyositis, diabetes, dysautonomia, eczema, encephalitis, endocarditis, endometriosis, endometritis, enteritis, enterocolitis, epicondylitis, epididymitis, fasciitis, fibromyalgia (fibrositis), gastritis, gastroenteritis, gingivitis, glomerulonephritis, Goodpasture's syndrome (and associated glomerulonephritis and pulmonary hemorrhage), Graves' disease, Guillain-Barré syndrome, Hashimoto's thyroiditis, hepatitis, hidradenitis suppurativa, hyperacute rejection of transplanted organs, idiopathic thrombocytopenic purpura (ITP), inflammatory bowel disease (including Crohn's disease and ulcerative colitis), inflammatory pelvic disease, interstitial cystitis, irritable bowel syndrome, juvenile arthritis, juvenile idiopathic arthritis, laryngitis, mastitis, meningitis, multiple vasculitides, myasthenia gravis, myelitis myocarditis, myocarditis, myositis, nephritis, neuromyotonia, oophoritis, opsoclonus-myoclonus syndrome, optic neuritis, orchitis, Ord's thyroiditis, osteitis, osteoarthritis, osteomyelitis, otitis, pancreatitis, Parkinson's disease, parotitis, pericarditis, peritonitis, pharyngitis, phlebitis, pleuritis, pneumonia, pneumonitis, primary biliary cirrhosis, proctitis, prostatitis, psoriasis (including psoriatic lesions in the skin), psoriatic arthritis, pyelonephritis, Reiter's syndrome, rheumatoid arthritis, rhinitis (including allergic rhinitis), rosacea, salpingitis, scleroderma, septic shock, sinusitis, Sjögren's syndrome, skin sunburn, skin sunburn, Still's disease, stomatitis, synovitis, Takayasu's arteritis, temporal arteritis, tendonitis, tissue graft rejection, tonsillitis, urethritis, urticaria, uveitis, uvitis, vaginitis, vasculitis (including antineutrophil cytoplasmic antibodies-associated vasculitis and immune complex mediated vasculitis), vulvitis, vulvodynia, warm autoimmune hemolytic anemia, and Wegener's granulomatosis.

### Combination Formulations and Uses Thereof

The compounds of the invention can be combined with one or more therapeutic agents. In particular, the therapeutic agent can be one that treats or prophylactically treats any disorder described herein, such as a B-cell related disorder, cancer, or an inflammatory or autoimmune disorder.

### Combination Formulations

In addition to the formulations described herein, one or more compounds of the invention can be used in combination with other therapeutic agents. For example, one or more compounds of the invention can be combined with another therapeutic agent. Exemplary therapeutic agent useful for this purpose include, without limitation, those described in U.S. Pat. Nos. 8,008,309; 7,943,618; 7,884,108; 7,868,018; 7,825,118; 7,642,255; 7,501,410; 7,405,295; 6,753,348; and 6,303,652. In particular embodiments, the compound of the invention is used in combination with an anti-cancer agent or an anti-inflammatory agent (e.g., a nonsteroidal anti-inflammatory drug, acetaminophen, a gold complex, a corticosteroid, or an immunosuppressant).

Non-limiting, exemplary anti-cancer agents include fludarabine, cyclophosphamide, methotrexate, rituximab, bendamustine, ofatumumab, dasatinib, U0126 ((2Z,3Z)-2,3-bis[amino-(2-aminophenyl)sulfanylmethylidene]butanedinitrile), PD98059 (2-(2-amino-3-methoxyphenyl)chromen-4-one), PD184352 (2-(2-chloro-4-iodoanilino)-N-(cyclopropylmethoxy)-3,4-difluorobenzamide), PD0325901 (N-[(2R)-2,3-dihydroxypropoxy]-3,4-difluoro-2-[(2-fluoro-4-iodophenyl)amino]-benzamide), ARRY-142886 (6-(4-bromo-2-chloroanilino)-7-fluoro-N-(2-hydroxyethoxy)-3-methylbenzimidazole-5-carboxamide), SB 239063 (trans-4-[4-(4-fluorophenyl)-5-(2-methoxy-4-pyrimidinyl)-1H-imidazol-1-yl]cyclohexanol), SP 600125 (anthra[1-9-cd]pyrazol-6(2H)-one), BAY 43-9006 (sorafenib or 4-[4-[[4-chloro-3(trifluoromethyl)phenyl]carbamoylamino]phenoxy]-N-methylpyridine-2-carboxamide), wortmannin, or LY 294002 (2-(4-morpholinyl)-8-phenyl-4H-1-benzopyran-4-one or a hydrochloride salt thereof). Additional non-limiting, exemplary classes of anti-cancer agents include other kinase inhibitors (e.g., a BTK inhibitor, e.g., PCI-32765 (1-[(3R)-3-[4-amino-3-(4-phenoxyphenyl)pyrazolo[3,4-d]pyrimidin-1-yl]piperidin-1-yl]prop-2-en-1-one), LOB 03-0110 ((3-(2-(3-(morpholinomethyl)phenyl)thieno[3,2-b]pyridin-7-ylamino)phenol), (−)-terreic acid ((1R,6S)-3-hydroxy-4-methyl-7-oxabicyclo[4.1.0]hept-3-ene-2,5-dione), LFM-A13 (2-cyano-N-(2,5-dibromophenyl)-3-hydroxy-2-butenamide), staurosporine, and dasatinib), topoisomerase I inhibitors (e.g., camptothecin and topotecan), topoisomerase II inhibitors (e.g., daunomycin and etoposide), alkylating agents (e.g., cyclophosphamide, melphalan, and carmustine (BCNU)), and anti-tubulin agents (e.g., taxol and vinblastine).

Non-limiting, exemplary anti-inflammatory agents include a nonsteroidal anti-inflammatory drug (an NSAID, e.g., non-specific and COX-2 specific cyclooxgenase enzyme inhibitors), acetaminophen, a gold complex, a corticosteroid, and an immunosuppressant. Non-limiting examples of NSAIDs include acemetacin, aspirin, celecoxib, deracoxib, diclofenac, diflunisal, ethenzamide, etodolac, etofenamate, etoricoxib, fenoprofen, flufenamic acid, flurbiprofen, hydroxychloroquine, ibuprofen, indomethacin, isoxicam, kebuzone, ketoprofen, ketorolac, lonazolac, lornoxicam, lumiracoxib, meclofenamic acid, mefenamic acid, meloxicam, metamizol, misoprostol, mofebutazone, naproxen, nabumetone, niflumic acid, piroxicam, oxaprozinpiroxicam, oxyphenbutazone, parecoxib, phenidone, phenylbutazone, piroxicam, propacetamol, propyphenazone, rofecoxib, salicylamide, salsalate, sulfasalazine, sulindac, suprofen, tiaprofenic acid, tenoxicam, tolmetin, valdecoxib, 4-(4-cyclohexyl-2-methyloxazol-5-yl)-2-fluorobenzenesulfonamide, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, and 2-(3,5-difluorophenyl)-3-[4-(methylsulfonyl)phenyl]-2-cyclopenten-1-one. Non-limiting examples of gold complexes include aurothioglucose, auranofin disodium aurothiomalate, sodium aurothiomalate, and sodium aurothiosulfate. Non-limiting examples of corticosteroids include cortisone, dexamethasone, methylprednisolone, prednisolone, prednisolone sodium phosphate, and prednisone. Non-limiting examples of immunosuppressants include alkylation agents (e.g., cyclophosphamide), antimetabolites (e.g., azathioprine, methotrexate, leflunomide, and mycophenolate mofetil), antibodies or antibody fragments or derivatives (e.g., an anti-C5 monoclonal antibody, such as eculizumab or pexelizumab; and a TNF antagonist, such as entanercept or infliximab, or fragments or derivatives of any of these), and macrolides (e.g., cyclosporine and tacrolimus).

Combination Therapies

A compound of the invention can be used alone or in combination with other agents that have BTK-inhibiting activity, or in combination with other types of treatment (which may or may not inhibit BTK) to treat, prevent, and/or reduce the risk of cancer, an inflammatory disorder, or other disorders that benefit from BTK inhibition. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered. Preferred dose ranges include, for example, between 0.05-15 mg/kg or between 0.5-15 mg/kg.

Alternatively, the dosage amount can be calculated using the body weight of the patient. For example, the dose of a compound, or pharmaceutical composition thereof, administered to a patient may range from 0.1-50 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### Diagnostic and Screening Assays

In addition to the above-mentioned therapeutic uses, a compound of the invention can also be used in diagnostic assays, screening assays, and as a research tool.

In diagnostic assays, a compound of the invention may be useful in identifying or detecting BTK activity.

In screening assays, a compound of the invention may be used to identify other compounds that inhibit BTK, for example, as first generation drugs. As research tools, the compounds of the invention may be used in enzyme assays and assays to study the extent of BTK activity. Such information may be useful, for example, for diagnosing or monitoring disease states or progression. In such assays, a compound of the invention may also be radiolabeled.

### BTK In Vitro Inhibition Assays

The compounds of the present invention have been found to exhibit BTK inhibition. Compounds may be examined for their efficacy in inhibiting kinase activity by a person skilled in the art, for example, by using the methods described in Example 1 and the other examples provided herein or by methods known in the literature (e.g., Mast Cells: Methods and Protocols (eds. G. Krishnaswamy and D. S. Chi), Methods in Molecular Biology, Series 315, Humana Press, pp. 175-192, 2006).

Inhibitory activity can be determined by any useful method. For example, inhibition can be determined by the effect of a test compound on BTK autophosphorylation. Btk and varying concentrations of the test compound can be included in a [γ-³²P]ATP-containing kinase buffer. Autophosphorylation can be analyzed by SDS/PAGE followed by electroblotting and autoradiography, where phosphorylated protein bands can be quantified by densitometry. These assays can be conducted without or with an exogenous substrate (e.g., glutathione S-transferase (GST)-IGα).

In another example, inhibitory activity can be determined by the effect of a test compound on BTK binding. For example, BTK can bind to protein kinase C (PKC) in vivo, where PKC in turn phosphorylates BTK. Accordingly, an exemplary assay to assess BTK-PKC binding includes incubating PKC or cell lysates having PKC (e.g., lysates from human mast cell lines) with glutathione S-transferase (GST)-Btk beads in the absence or presence of the test compound. Then, the extent of Btk-bound PKC can be detected by any useful manner, such as by SDS/PAGE followed by immunoblotting with anti-PKC (MC5) and/or anti-BTK antibodies.

Further examples include use of cellular assays, such as by determining the effect of a test compound on cellular activation. For example, stimulated lymphoid, myeloid, or mast cells (e.g., cells stimulated with a signaling molecule, such as erythropoietin or an antigen, such as IgE) can be incubated with a test compound, and the activation of particular compounds or proteins can be measured. Exemplary compounds and proteins include histamine, leukotriene, cytokines, PKC, Janus tyrosine kinase 2 (Jak2), erythropoietin receptor (EpoR), Stat5, protein kinase B (PKB), and/or mitogen activating protein kinase (Erk1/2). In another example, as activated Btk can be phosphorylated at tyrosine 223 (Y223) and/or tyrosine 551 (Y551), cellular assays can be conducted by staining P-Y223 or P-Y551-positive cells in a population of cells (e.g., by phosphorylation-specific immunochemical staining followed by FACS analysis).

As BTK is a tyrosine kinase, additional useful assays include any tyrosine kinase assay. In particular, commercially available assays include kinase assays that detect formation of ADP, e.g., with luminescent detection, such as in an ADP-Glo™ Kinase Assay (Promega Corp., Madison, Wisc.). Dose response curves can be obtained by incubating BTK with a substrate (e.g., ATP or a binding partner, such as PKC) and increasing (e.g., logarithmically increasing) the concentration of a test compound. In addition, a detectable agent (e.g., a luminescent probe, such as a luciferase/luciferin reaction that measures ATP) can be used to correlate kinase activity (e.g., ATP-to-ADP conversion) with the concentration of the test compound. These data can be used to construct a dose response curve, where IC₅₀ is the concentration of the test compound that provides about 50% inhibition.

The following non-limiting examples are illustrative of the present invention.

### EXAMPLES

### Example 1

### Synthesis of Compound 1

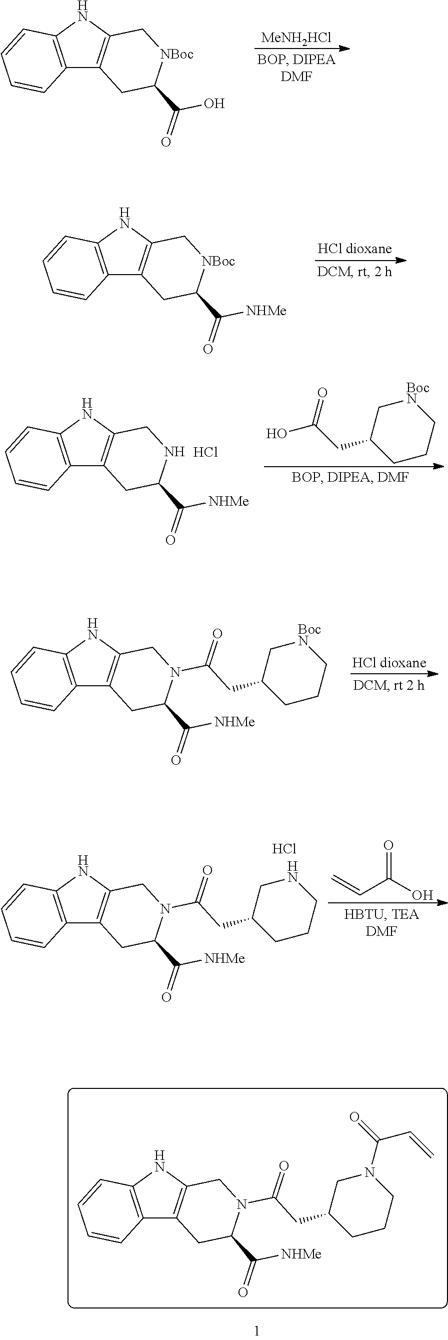

### Experimental Procedure:

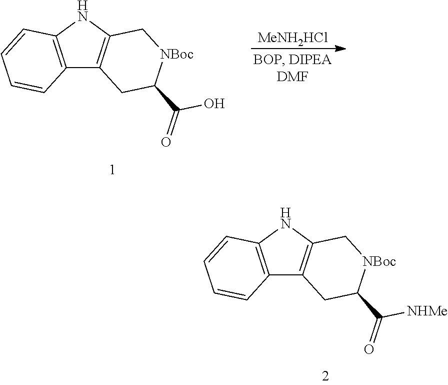 [SMILES: "MeNH2HCl" ; CC(C)(C)OC(=O)N1Cc2[nH]c3ccccc3c2C[C@@H]1C(=O)O ; CNC(=O)[C@H]1Cc2c([nH]c3ccccc23)CN1C(=O)OC(C)(C)C]

Synthesis of tert-butyl (R)-3-(methylcarbamoyl)-1,3,4,9-tetrahydro-2H-pyrido[3,4-b]indole-2-carboxylate: To a solution of (R)-2-(tert-butoxycarbonyl)-2,3,4,9-tetrahydro-1H-pyrido[3,4-b]indole-3-carboxylic acid (200 mg, 0.63 mmol) and (Benzotriazol-1-yloxy)tris(dimethylamino)phosphonium hexafluorophosphate (BOP) (420 mg, 0.94 mmol) in DMF (3 mL), was added methylamine hydrochloride (84 mg, 1.26 mmol) and diisopropylethylamine (0.39 mL, 1.90 mmol). The mixture was stirred at room temperature overnight, diluted with DCM (5 mL), washed with water (3 mL×3), dried over sodium sulfate and concentrated. Product was purified using silica gel column chromatography with 0-50% THF in DCM to give the title compound as a white solid (175 mg).

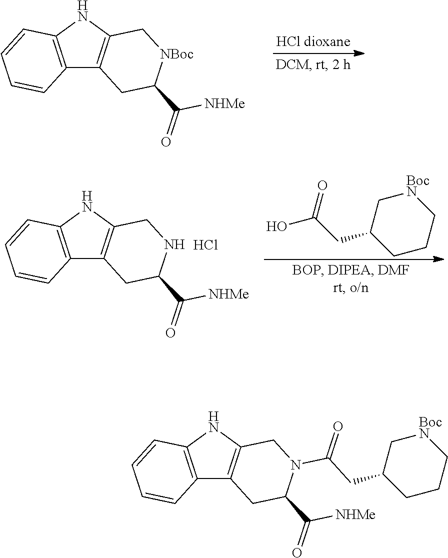 [SMILES: CNC(=O)[C@H]1Cc2c([nH]c3ccccc23)CN1C(=O)OC(C)(C)C ; CNC(=O)[C@H]1Cc2c([nH]c3ccccc23)CN1 ; Cl ; CC(C)(C)OC(=O)N1CCC[C@H](CC(=O)O)C1 ; CNC(=O)[C@H]1Cc2c([nH]c3ccccc23)CN1C(=O)C[C@H]1CCCN(C(=O)OC(C)(C)C)C1]

Synthesis of tert-butyl (R)-3-(2-((R)-3-(methylcarbamoyl)-1,3,4,9-tetrahydro-2H-pyrido[3,4-b]indol-2-yl)-2-oxoethyl)piperidine-1-carboxylate: To a solution of tert-butyl (R)-3-(methylcarbamoyl)-1,3,4,9-tetrahydro-2H-pyrido[3,4-b]indole-2-carboxylate (175 mg from step 1) in DCM (5 mL) was added 4.0 M HCl in dioxane (3.0 mL). The mixture was stirred at room temperature for 2 hrs before diethylether (10 mL) was added. Solid was collected to give the deprotected compound (150 mg) as an HCl salt. To a solution of (R)-2-(1-(tert-butoxycarbonyl)piperidin-3-yl)acetic acid (109 mg, 0.45 mmol) and BOP (199 mg, 0.45 mmol) in DMF (3 mL) was added the product above (100 mg, 0. 38 mmol) and diisopropylethylamine (0.26 mL, 1.52 mmol). The mixture was stirred at room temperature overnight, diluted with DCM (5 mL), washed with water (3 mL×3), dried over sodium sulfate and concentrated. Product was purified using silica gel column chromatography with 0-50% THF in DCM to give the title compound as a brown oil. It was used in the next step without further purification.

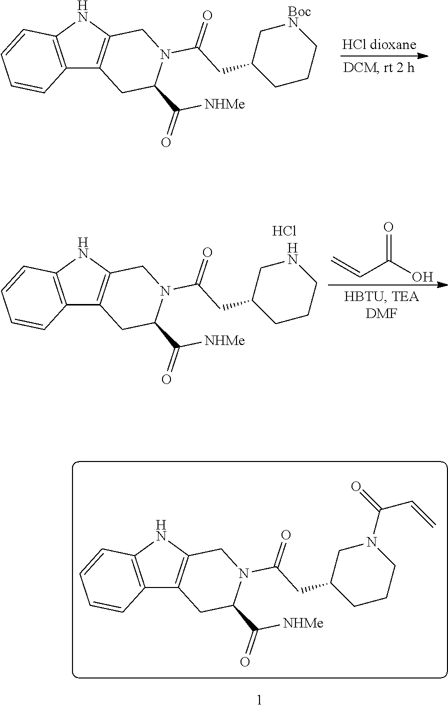

Synthesis of (R)-2-(2-((R)-1-acryloylpiperidin-3-yl)acetyl)-N-methyl-2,3,4,9-tetrahydro-1H-pyrido[3,4-1]indole-3-carboxamide (1): To a solution of tert-butyl (R)-3-(2-((R)-3-(methylcarbamoyl)-1,3,4,9-tetrahydro-2H-pyrido[3,4-b]indol-2-yl)-2-oxoethyl)piperidine-1-carboxylate (from step 3) in DCM (5 mL) was added 4.0M HCl in dioxane (1.5 mL). The mixture was stirred at RT for 2 hrs before diethylether (10 mL) was added. Solid was collected to give deprotected compound as an HCl salt. To a solution of the deprotected intermediate (50 mg, 0.13 mmol) in DMF (2 mL) was added acrylic acid (0.13 mmol) and HBTU (66 mg, 0.17 mmol) followed by the addition of TEA (0.083 mL, 0.60 mmol). The mixture was stirred at RT overnight, diluted with DCM (5 mL), washed with water (3 mL×3), dried over sodium sulfate and concentrated. Product was purified by silica gel column chromatography with 0-10% methanol in DCM to give 1 as a yellow solid (17 mg). LC-MS (M+H)⁺=409.
¹H NMR (300 MHz, DMSO-d₆) δ10.78 (d, 1H), 7.88-7.77 (m, 1H), 7.40 (d, J=7.5 Hz, 1H), 7.31-7.27 (m, 1H), 7.06-6.90 (m, 2H), 6.85-6.72 (m, 1H), 5.50-5.70 (m, 2H), 5.20-4.50 (m, 2H), 4.24-3.97 (m, 2H), 3.63-3.59 (m, 2H), 3.10-3.05 (m, 5H), 1.86-1.84 (m, 2H), 1.63 (bs, 1H), 1.12-1.20 (m, 5H).

### Example 2

### Synthesis of Compound 2

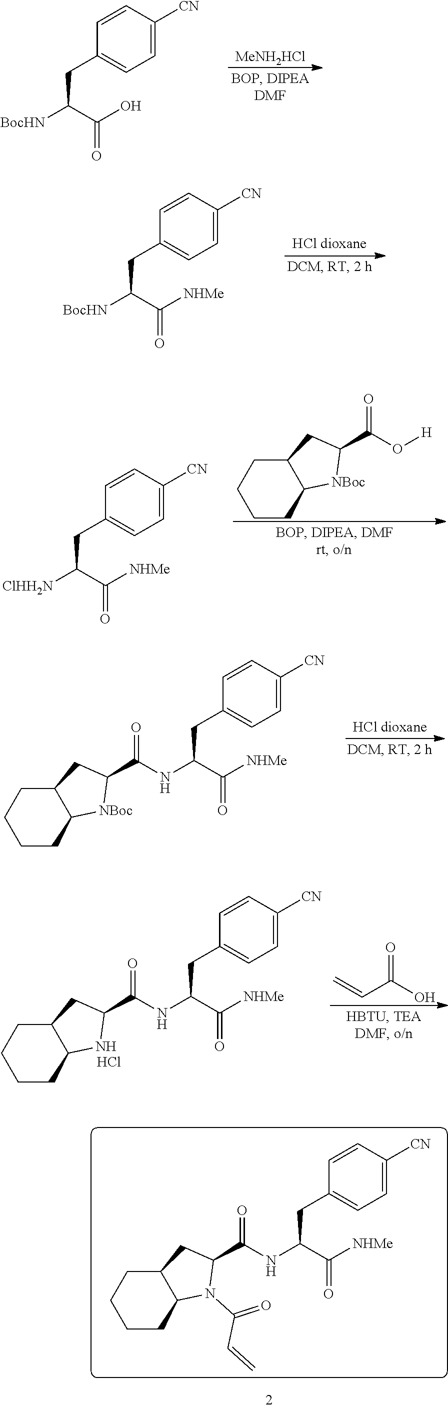

### Experimental Procedure

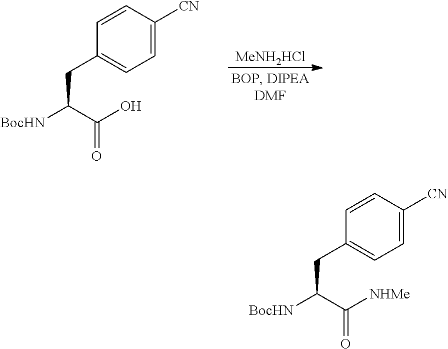 [SMILES: "MeNH2HCl" ; CC(C)(C)OC(=O)N[C@@H](Cc1ccc(C#N)cc1)C(=O)O ; CNC(=O)[C@H](Cc1ccc(C#N)cc1)NC(=O)OC(C)(C)C]

Synthesis of tert-butyl (S)-(3-(4-cyanophenyl)-1-(methylamino)-1-oxopropan-2-yl)carbamate: To a solution of (S)-2-((tert-butoxycarbonyl)amino)-3-(4-cyanophenyl)propanoic acid (200 mg, 0.69 mmol) and (Benzotriazol-1-yloxy)tris(dimethylamino)phosphonium hexafluorophosphate (BOP) (457 mg, 1.03 mmol) in DMF (2 mL) was added methylamine hydrochloride (92 mg, 1.38 mmol) and diisopropylethylamine (0.36 mL, 2.07 mmol). The mixture was stirred at room temperature overnight, diluted with DCM (5 mL), washed with water (3 mL×3), dried over sodium sulfate and concentrated. Product was purified using silica gel column chromatography with 0-50% THF in DCM to give the title compound as a white solid (160 mg).

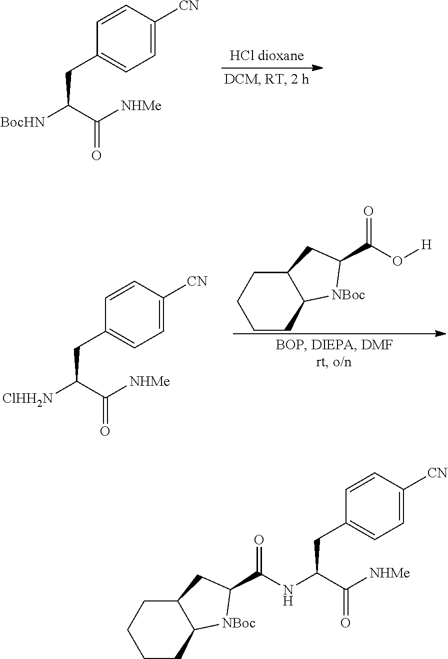

Synthesis of tert-butyl (2S,3aS,7aS)-2-(((S)-3-(4-cyanophenyl)-1-(methylamino)-1-oxopropan-2-yl)carbamoyl)octahydro-1H-indole-1-carboxylate: To a solution of tert-butyl (S)-(3-(4-cyanophenyl)-1-(methylamino)-1-oxopropan-2H)carbamate (160 mg from step 1) in DCM (5 mL) was added 4.0M HCl in dioxane (3.0 mL). The mixture was stirred at room temperature for 2 hrs before diethylether (10 mL) was added. Solid was collected to give the product (150 mg) as an HCl salt. To a solution of Boc-L-octahydroindole-2-carboxylic acid (134 mg, 0.5 mmol) and BOP (221 mg, 0.5 mmol) in DMF (3 mL) was added the product above (100 mg, 0. 42 mmol) and diisopropylethylamine (0.29 mL, 1.68 mmol). The mixture was stirred at room temperature overnight, diluted with DCM (5 mL), washed with water (3 mL×3), dried over sodium sulfate and concentrated. Product was purified using silica gel column chromatography with 0-50% THF in DCM to give the title compound as a white solid (180 mg).

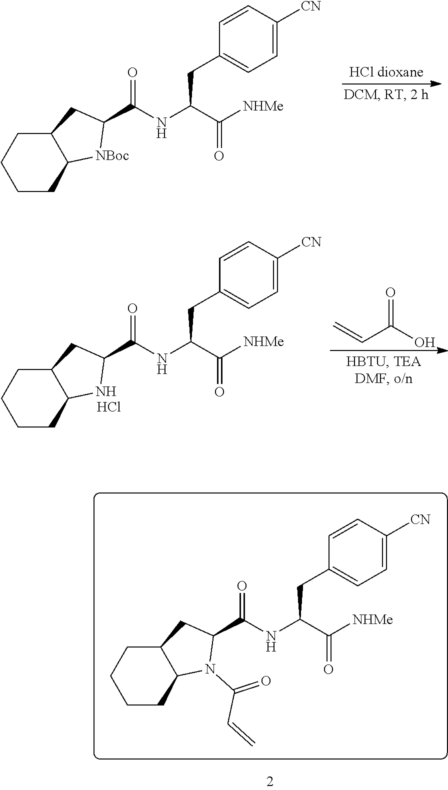

Synthesis of (2S,3aS,7aS)-1-acryloyl-N-((S)-3-(4-cyanophenyl)-1-(methylamino)-1-oxopropan-2-yl)octahydro-1H-indole-2-carboxamide (2): To a solution of tert-butyl (2S,3aS,7aS)-2-(((S)-3-(4-cyanophenyl)-1-(methylamino)-1-oxopropan-2-yl)carbamoyl)octahydro-1H-indole-1-carboxylate (180 mg) in DCM (5 mL), was added 4.0M HCl in dioxane (1.5 mL). The mixture was stirred at RT for 2 hrs before diethylether (10 mL) was added. Solid was collected to give product as an HCl salt. To a solution of the product (30 mg, 0.077 mmol) in DMF (2 mL) was added a solution of acrylic acid (0.12 mmol) in DMF (100 uL) and HBTU (35 mg, 0.092 mmol), followed by the addition of TEA (0.043 mL, 0.31 mmol). The mixture was stirred at room temperature overnight, diluted with DCM (5 mL), washed with water (3 mL×3), dried over sodium sulfate and concentrated. Product was purified using silica gel column chromatography with 0-10% methanol in DCM to give 2 as a white solid (10 mg) after collecting only the pure fractions from the column. LC-MS (M+H)⁺=409.
¹H NMR (300 MHz, CDCl₃) δ7.53 (d, J=8.1 Hz, 2H), 7.30 (d, 2H), 6.88 (d, J=8.1 Hz, 1H), 6.74 (bs, 1H), 6.37-6.39 (m, 2H), 5.78-5.82 (m, 1H), 4.74-4.77 (m, 1H), 4.47 (t, 1H), 3.82-3.85 (m, 1H), 3.44-3.13 (m, 2H), 2.82 (d, J=4.8 Hz, 3H), 2.35-2.01 (m, 3H), 1.81-1.10 (m, 8H).

### Example 3

### Synthesis of Compound 11

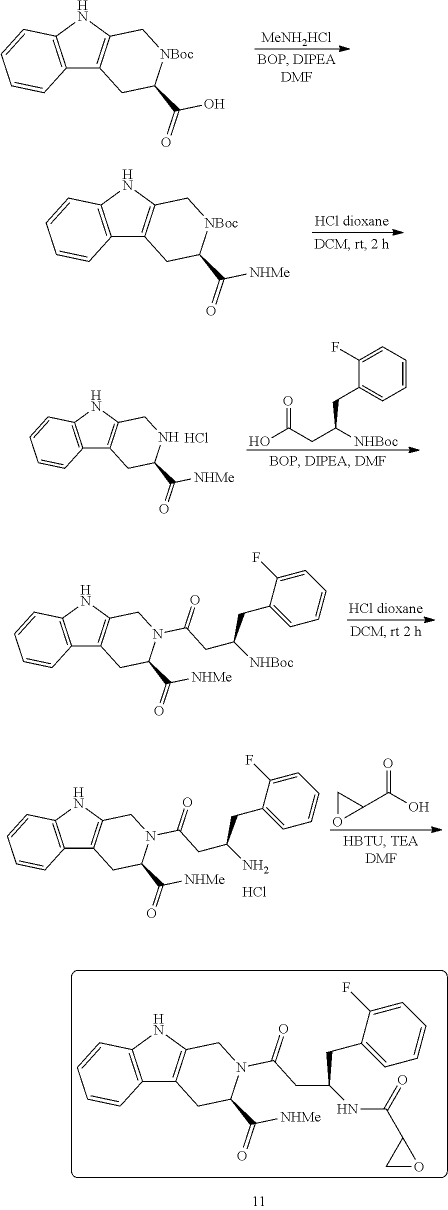

### Example 4

### Determination of BTK inhibition Activity

The compounds were assayed for BTK inhibition activity using the Invitrogen™ LanthaScreen® Kinase Binding Assay. In short, the compounds were tested for their ability to displace a tracer (in this case Invitrogen™ Kinase Tracer 236) from the active site of BTK. The BTK protein used in the assay was labeled with europium (Eu), and so displacement was conveniently detected as a loss of Eu-to-tracer FRET (fluorescence resonance energy transfer) signal using a plate reader equipped to measure TR-FRET (time resolved FRET). This displacement assay is commonly used to characterize kinase inhibitors and it is predictive of kinase inhibitory activity.

Several of the compounds were also tested directly for kinase inhibitory activity using the Invitrogen™ Omnia® assay. The Omnia® assay is a real time kinetic assay that uses a phosphate-induced fluorophore to detect transfer of phosphate from ATP to a peptide. Inhibition of kinase activity in this assay reduces the rate of fluorescence increase. Compounds tested in both assays demonstrated similar IC₅₀ values. More details and experimental protocols for both assays can be found at invitrogen.com.

Determination of IC₅₀ Values

Various compounds of the invention (i.e., compounds of formula (I) or (Ia)) were assayed for BTK inhibition activity, as described above, and possessed IC₅₀ values less than 1.0 μM. In some embodiments, the compounds possessed IC₅₀ values less than 0.9 μM, less than 0.8 μM, less than 0.5 μM, less than 0.3 μM, less than 0.2 μM, less than 0.1 μM, less than 0.09 μM, less than 0.08 μM, less than 0.05 μM, less than 0.04 μM, less than 0.03 μM, less than 0.025 μM, less than 0.015 μM, less than 0.01 μM, less than 0.005 μM, less than 0.002 μM, less than 0.0015 μM, or less than 0.001 μM. In some embodiments, the compounds possessed IC₅₀ values from 0.0001 μM to 0.9 μM (e.g., from 0.0001 μM to 0.8 μM, from 0.0001 μM to 0.5 μM, from 0.0001 μM to 0.3 μM, from 0.0001 μM to 0.2 μM, from 0.0001 μM to 0.1 μM, from 0.0001 μM to 0.09 μM, from 0.0001 μM to 0.08 μM, from 0.0001 μM to 0.05 μM, from 0.0001 μM to 0.04 μM, from 0.0001 μM to 0.03 μM, from 0.0001 μM to 0.025 μM, from 0.0001 μM to 0.015 μM, from 0.0001 μM to 0.01 μM, from 0.0001 μM to 0.005 μM, 0.0002 μM to 0.9 μM, from 0.0002 μM to 0.8 μM, from 0.0002 μM to 0.5 μM, from 0.0002 μM to 0.3 μM, from 0.0002 μM to 0.2 μM, from 0.0002 μM to 0.1 μM, from 0.0002 μM to 0.09 μM, from 0.0002 μM to 0.08 μM, from 0.0002 μM to 0.05 μM, from 0.0002 μM to 0.04 μM, from 0.0002 μM to 0.03 μM, from 0.0002 μM to 0.025 μM, from 0.0002 μM to 0.015 μM, from 0.0002 μM to 0.01 μM, from 0.0002 μM to 0.005 μM, 0.0005 μM to 0.9 μM, from 0.0005 μM to 0.8 μM, from 0.0005 μM to 0.5 μM, from 0.0005 μM to 0.3 μM, from 0.0005 μM to 0.2 μM, from 0.0005 μM to 0.1 μM, from 0.0005 μM to 0.09 μM, from 0.0005 μM to 0.08 μM, from 0.0005 μM to 0.05 μM, from 0.0005 μM to 0.04 μM, from 0.0005 μM to 0.03 μM, from 0.0005 μM to 0.025 μM, from 0.0005 μM to 0.015 μM, from 0.0005 μM to 0.01 μM, from 0.0005 μM to 0.005 μM, from 0.0005 μM to 0.002 μM, from 0.0005 μM to 0.0015 μM, or from 0.0005 μM to 0.001 μM).

### Example 5

### Determination of Compound Off-Rate

Biotinylated BTK (20 nM final concentration) was premixed with Europium labeled streptavidin (10 nM final concentration) and the complex was incubated for 18 hours at room temperature with compounds at various concentrations in assay buffer (25 mM HEPES, 10 mM MgCl₂, 0.5 mg/ml bovine serum albumin, 1% DMSO) in a final volume of 100 μL. Following this incubation, 2 μL of the BTK/compound mixture was diluted 10-fold into 18 μL of buffer containing 30 nM Kinase Tracer 236 (ThermoFisher), an Alexa Fluor® conjugated non-covalent compound that binds to multiple kinase active sites including BTK. The final concentration of compound after dilution was at 2-30 fold below its IC₅₀ as determined by a competition binding assay also using Tracer 236. The TR-FRET signal generated by the proximity of the Eu-streptavidin to Tracer 236 when both bind simultaneously to BTK was read in a Tecan Infinite M1000 Pro plate reader (excitation 620 nm, emission 665 nm) at regular intervals over 6 hours. An increase in TR-FRET signal over time should be closely related to the off-rate of the compound after dilution. Controls without BTK and with BTK only, not including compound, were also included, setting the low and high ranges of the assay respectively. Control assays with non-covalent BTK inhibitors resulted in a steady increase in TR-FRET signal over time indicating that these compounds could be displaced by the tracer. A covalent BTK inhibitor, ibrutinib, completely prevented the increase in TR-FRET signal indicating that, consistent with its covalent mechanism of action, ibrutinib could not be displaced by the tracer.

### Other Embodiments

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

What is claimed:

1. A compound having the structure of Formula I:
A—B—C Formula I
wherein A is 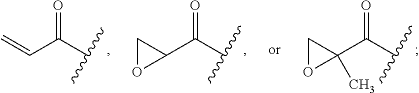
B has the structure: 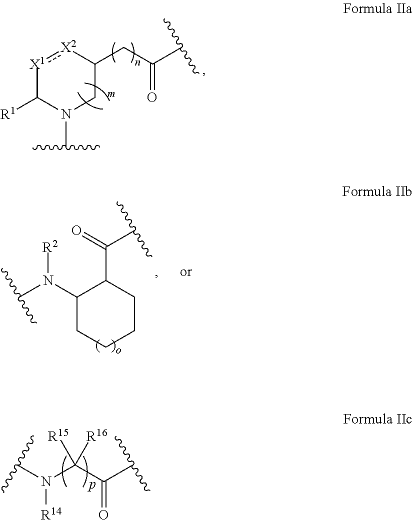
wherein B is conjugated to A at the nitrogen and to C at the carbonyl;
the dotted line is an optional double bond;
m is O or 1;
n is 0, 1, or 2;
o is 0 or 1;
p is 1 or 2;
R¹ is hydrogen or combines with R³ or R⁴ to form a 5- to 6-membered carbocyclic ring;
R² is hydrogen or C₁-C₆ alkyl;
X¹ is S or CR³R⁴, wherein R³ and R⁴ are each, independently, hydrogen or combine with R¹ to form a 5- to 6-membered carbocyclic ring, wherein if a double bond is present, then R⁴ is absent; and
X² is CR⁵R⁶, wherein R⁵ and R⁶ are each, independently, hydrogen or C₁-C₆ alkyl, wherein if a double bond is present, R⁶ is absent;
R¹⁴ is hydrogen or C₁-C₆ alkyl;
each R¹⁵ and R¹⁶ is, independently, optionally substituted C₁-C₆ heteroalkyl, optionally substituted C₆-C₁₀ aryl; or optionally substituted C₁-C₆ alkyl C₆-C₁₀ aryl; and
C has the structure: 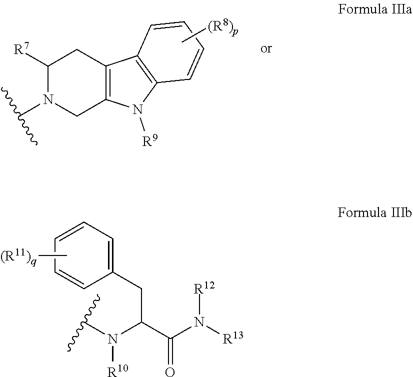
wherein p is 0, 1, 2, 3, or 4;
q is 0, 1, 2, 3, 4, or 5;
R⁷ is hydrogen or amido;
each R⁸ and R¹¹ is, independently hydrogen, hydroxy, C₁-C₆ alkyl, cyano, or halo; and
R⁹, R¹⁰, R¹², and R¹³ are each, independently, hydrogen or C₁-C₆ alkyl,
or a pharmaceutically acceptable salt thereof.

2. The compound of claim 1, wherein B has the structure of Formula IIa: 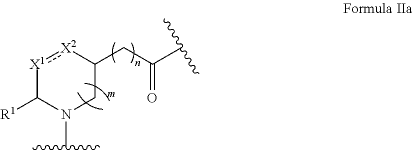
wherein B is conjugated to A at the nitrogen and to C at the carbonyl;
the dotted line is an optional double bond;
m is 0 or 1;
n is 0, 1, or 2;
R¹ is hydrogen or combines with R³ or R⁴ to form a 5- to 6-membered carbocyclic ring;
X¹ is S or CR³R⁴, wherein R³ and R⁴ are each, independently, hydrogen or combine with R¹ to form a 5- to 6-membered carbocyclic ring, wherein if a double bond is present then R⁴ is absent; and
X² is CR⁵R⁶, wherein R⁵ and R⁶ are each, independently, hydrogen or C₁-C₆ alkyl, wherein if a double bond is present, R⁶ is absent.

3. The compound of claim 1 or 2, wherein m is 1.

4. The compound of claim 3, wherein R¹ is hydrogen.

5. The compound of claim 3 or 4, wherein X¹ is CR³R⁴.

6. The compound of claim 5, wherein R³ and R⁴ are both hydrogen.

7. The compound of any one of claims 3 to 6, wherein X² is CR⁵R⁶.

8. The compound of claim 7, wherein R⁵ and R⁶ are both hydrogen.

9. The compound of any one of claims 3 to 8, wherein n is 1.

10. The compound of any one of claims 3 to 9, wherein B has the structure:

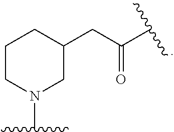 [SMILES: *C(=O)CC1CCCN(*)C1]

11. The compound of claim 10, wherein B has the structure:

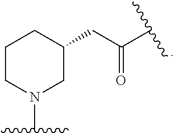 [SMILES: *C(=O)C[C@H]1CCCN(*)C1]

12. The compound of claim 2, wherein m is 0.

13. The compound of claim 12, wherein n is 2.

14. The compound of claim 13, wherein R¹ is hydrogen.

15. The compound of claim 13 or 14, wherein X¹ is CR³R⁴.

16. The compound of claim 15, wherein R³ and R⁴ are both hydrogen.

17. The compound of any one of claims 13 to 16, wherein X² is CR⁵R⁶.

18. The compound of claim 17, wherein R⁵ and R⁶ are both hydrogen.

19. The compound of any one of claims 13 to 18, wherein B has the structure:

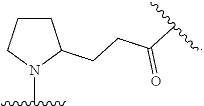 [SMILES: *C(=O)CCC1CCCN1*]

20. The compound of claim 12, wherein n is 0.

21. The compound of claim 20, wherein R¹ is hydrogen.

22. The compound of claim 21, wherein X¹ is S.

23. The compound of claim 21 or 22, wherein X² is CR⁵R⁶.

24. The compound of claim 23, wherein R⁵ and R⁶ are both methyl.

25. The compound of any one of claims 20 to 24, wherein B has the structure:

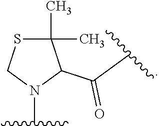 [SMILES: *C(=O)C1N(*)CSC1(C)C]

26. The compound of claim 25, wherein B has the structure:

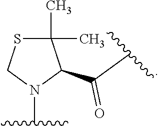 [SMILES: *C(=O)[C@H]1N(*)CSC1(C)C]

27. The compound of claim 21, wherein B has the structure:

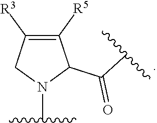

28. The compound of claim 27, wherein R³ and R⁵ are both hydrogen.

29. The compound of claim 21, wherein X² is CR⁵R⁶.

30. The compound of claim 29, wherein R⁵ and R⁶ are both hydrogen.

31. The compound of claim 29 or 30, wherein X¹ is CR³R⁴.

32. The compound of claim 31, wherein R⁴ is hydrogen.

33. The compound of anyone of claims 29 to 32, wherein R¹ and R³ combine to form a 5- or 6-membered carbocyclic ring.

34. The compound of any one of claims 29 to 33, wherein B has the structure:

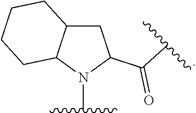 [SMILES: *C(=O)C1CC2CCCCC2N1*]

35. The compound of claim 34, wherein B has the structure:

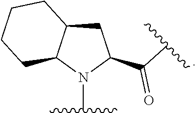 [SMILES: *C(=O)[C@@H]1C[C@@H]2CCCC[C@@H]2N1*]

36. The compound of claim 1, wherein B has the structure of Formula IIb: 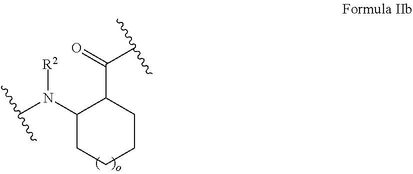
wherein B is conjugated to A at the nitrogen and to C at the carbonyl;
o is 0 or 1; and
R² is hydrogen or C₁-C₆ alkyl.

37. The compound of claim 36, wherein o is 0.

38. The compound of claim 36 or 37, wherein R² is hydrogen.

39. The compound of claim 1 wherein B has the structure of Formula IIc: 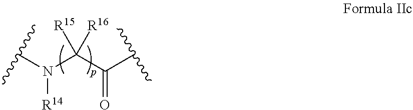
wherein B is conjugated to A at the nitrogen and to C at the carbonyl;
p is 1 or 2;
R¹⁴ is hydrogen or C₁-C₆ alkyl; and
each R¹⁵ and R¹⁶ is, independently, optionally substituted C₁-C₆ heteroalkyl, optionally substituted C₆-C₁₀ aryl; or optionally substituted C₁-C₆ alkyl C₆-C₁₀ aryl.

40. The compound of claim 39, wherein R¹⁴ is hydrogen.

41. The compound of claim 39 or 40, wherein p is 1.

42. The compound of any one of claims 39 to 41, wherein R¹⁵ is hydrogen.

43. The compound of any one of claims 39 to 42, wherein R¹⁶ is optionally substituted C₆-C₁₀ aryl.

44. The compound of claim 43, wherein optionally substituted C₆-C₁₀ aryl is phenyl.

45. The compound of any one of claims 39 to 42, wherein R¹⁶ is optionally substituted C₁-C₆ heteroalkyl.

46. The compound of claim 45, wherein optionally substituted C₁-C₆ heteroalkyl is

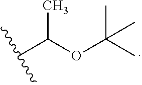 [SMILES: *C(C)OC(C)(C)C]

47. The compound of claim 39, wherein B has the structure:

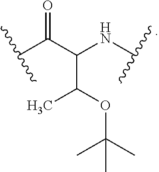 [SMILES: *NC(C(*)=O)C(C)OC(C)(C)C]

48. The compound of claim 39, wherein B has the structure:

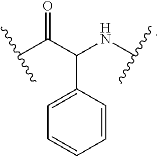 [SMILES: *NC(C(*)=O)c1ccccc1]

49. The compound of claim 39 or 40, wherein p is 2.

50. The compound of claim 49, wherein R¹⁵ is hydrogen.

51. The compound of claim 49 or 50, wherein R¹⁶ is optionally substituted C₁-C₆ alkyl C₆-C₁₀ aryl.

52. The compound of claim 51, wherein optionally substituted C₁-C₆ alkyl C₆-C₁₀ aryl is 2-fluoro-benzyl.

53. The compound of any one of claims 49 to 52, wherein B has the structure:

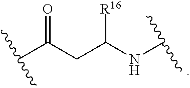

54. The compound of claim 53, wherein B has the structure:

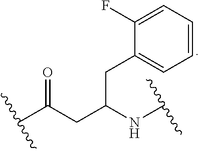 [SMILES: *NC(CC(*)=O)Cc1ccccc1F]

55. The compound of any one of claims 1 to 54, wherein C has the structure of Formula IIIa: 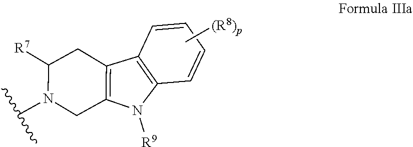
wherein p is 0, 1, 2, 3, or 4;
R⁷ is hydrogen or amido;
each R⁸ is, independently hydrogen, hydroxy, C₁-C₆ alkyl, cyano, or halo; and
R⁹ is hydrogen or C₁-C₆ alkyl.

56. The compound of claim 55, wherein p is 0.

57. The compound of claim 55 or 56, wherein R⁹ is hydrogen.

58. The compound of any one of claims 55 to 57, wherein R⁷ is amido.

59. The compound of any one of claims 55 to 58, wherein C has the structure:

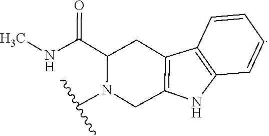 [SMILES: *N1Cc2[nH]c3ccccc3c2CC1C(=O)NC]

60. The compound of claim 59, wherein C has the structure:

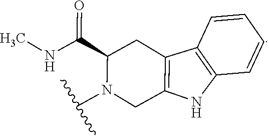 [SMILES: *N1Cc2[nH]c3ccccc3c2C[C@@H]1C(=O)NC]

61. The compound of any one of claims 1 to 54, wherein C has the structure: 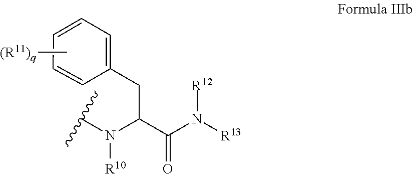
wherein q is 0, 1, 2, 3, 4, or 5;
each R¹¹ is, independently hydrogen, hydroxy, C₁-C₆ alkyl, cyano, or halo; and
R¹² and R¹³ are each, independently, hydrogen or C₁-C₆ alkyl.

62. The compound of claim 61, wherein R¹⁰ is hydrogen.

63. The compound of claim 60 or 61, wherein R¹² is hydrogen.

64. The compound of any one of claims 60 to 63, wherein R¹³ is C₁-C₆ alkyl.

65. The compound of claim 64, wherein C₁-C₆ alkyl is methyl.

66. The compound of any one of claims 60 to 65, wherein q is 1.

67. The compound of any one of claims 60 to 66, wherein C has the structure:

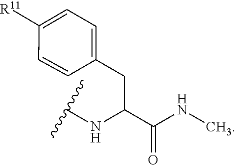

68. The compound of claim 67, wherein C has the structure:

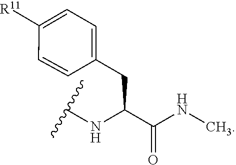

69. The compound of any one of claims 61 to 68, wherein R¹¹ is cyano.

70. The compound of any one of claims 1 to 69, wherein A is

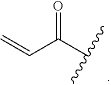 [SMILES: *C(=O)C=C]

71. The compound of any one of claims 1 to 69, wherein A is

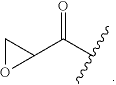 [SMILES: *C(=O)C1CO1]

72. The compound of any one of claims 1 to 69, wherein A is

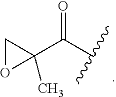 [SMILES: *C(=O)C1(C)CO1]

73. A compound having the structure of any one of compounds 1-11 in Table 1.

74. A pharmaceutical composition comprising a compound of any one of claims 1 to 73 and a pharmaceutically acceptable excipient.

75. A method of inhibiting Bruton's tyrosine kinase, said method comprising contacting a cell with a compound of any one of claims 1 to 73.

76. A method of treating a B-cell associated disease or a mast cell associated disease in a subject in need thereof, said method comprising administering an effective amount of a compound of any one of claims 1 to 73 or a pharmaceutically acceptable salt thereof, or a composition of claim 74 to said subject.

77. The method of claim 76, wherein said B-cell associated disease or said mast cell associated disease is cancer, an inflammatory disorder, or an autoimmune disorder.

78. A method of treating cancer in a subject in need thereof, said method comprising administering an effective amount of a compound of any one of claims 1 to 73 or a pharmaceutically acceptable salt thereof, or a composition of claim 74 to said subject.

79. The method of claim 78, wherein said cancer is leukemia, lymphoma, myeloma, or a pancreatic neoplasm.

80. The method of claim 78 or 79, wherein said cancer is non-Hodgkin lymphoma, B-cell lymphoma, chronic lymphocytic leukemia, small lymphocytic lymphoma, pancreatic insulinoma, pancreatic glucagonoma, or pancreatic gastrinoma.

81. A method of treating an inflammatory or autoimmune disorder in a subject in need thereof, said method comprising administering an effective amount of a compound of any one of claims 1 to 73 or a pharmaceutically acceptable salt thereof, or a composition of claim 74 to said subject.

82. The method of claim 81, wherein said inflammatory or autoimmune disorder is rheumatoid arthritis, systemic lupus erythematosus, multiple sclerosis, idiopathic thrombocytopenic purpura, glomerulonephritis, autoimmune-mediated hemolytic anemia, immune complex mediated vasculitis, or psoriasis.
